(19) United States
(12) Patent Application Publication
(10) Pub. No.: US 2006/0009912 A1
(43) Pub. Date: Jan. 12, 2006

(54) Microbiological information system

(75) Inventors: Henricus Matheus Wilhelmus Thijsen, Houten (NL); Roy Christiaan Montijn, Amsterdam (NL); Frank Henri Johan Schuren, Veenendaal (NL)

Correspondence Address: NORRIS, MCLAUGHLIN & MARCUS, P.A., 875 THIRD AVE, 18TH FLOOR, NEW YORK, NY, 10022, US

(73) Assignee: Nederlandse Oranisatie Voor Toegepastnatuurwetenschappelijk Onderzoek TNO, Delft (NL)

(21) Appl. No.: 10/512,526

(22) PCT Filed: Apr. 24, 2003
(86) PCT No.: PCT/NL03/00302

§ 371 Date: Oct. 20, 2004

(30) Foreign Application Priority Data
Apr. 25, 2002 (NL) 1020471

(51) Int. Cl. G06F 19/00; G01N 33/48; G01N 33/50

(52) U.S. Cl. 702/19

(57) ABSTRACT

The present invention relates to an apparatus and a method for the classification of microorganisms. In particular, the invention relates to an apparatus and method for the classification of microorganisms with annotation of clinically and/or industrially relevant information. An apparatus according to the invention comprises a measuring apparatus for classifying a microorganism according to distinctive features, a database containing clinically and/or industrially relevant information of a plurality of microorganisms, and a calculation unit for comparing the results from the measuring apparatus to the information in the database for the annotation of the clinically and/or industrially relevant information to the microorganism, and in which the measuring apparatus, the database and the data analysis apparatus are provided with input and output means.

The invention relates to an apparatus for the classification of microorganisms. In particular, the invention relates to an apparatus for the classification of microorganisms with annotation of clinically and/or industrially relevant information.

Antibiotic resistance of infectious microorganisms is an important problem in microbiology, medicine and health care. Antibiotic resistance has its origin in the evolution of microorganisms and the changes and spread of genetic material in and among microorganisms. The resistance is strongly affected by the use of antibiotics and spreads inter alia in hospitals and through our food (Davies, 1997).

In many cases, plasmids and transposons are the vehicle for transfer of genetic information, such as complete genes, among microorganisms. In this manner, genes for, for instance, antibiotic resistance of an organism can be transferred to another—not necessarily taxonomically related—organism.

Currently, many antibiotic resistance genes are known. For instance, it is known that resistance to the β-lactam penicillin can be encoded by inter alia pbp genes (penicillin binding protein) and by the bla operon (beta-lactamase). The genes responsible for antibiotic resistance are chromosome or plasmid-localized.

In many species of microorganisms, resistant strains are currently known, including strains from the genera Streptococcus, Staphylococcus, Campylobacter, Haemophilus and Mycobacterium. In fact, all microorganisms are capable of producing antibiotic-resistant strains.

For health care, especially those microorganisms are of interest that have an infectious nature, that is, they cause an infection in the human body which may or may not be contagious. It is particularly these clinically relevant microorganisms that increasingly show resistance to existing antimicrobial agents. A good example of this is MRSA, the meticillin-resistant form of the Staphylococcus aureus bacterium.

In detection and identification research of microorganisms and for determining an antibiotic susceptibility pattern, routine methods are used in the clinic. For the detection of a microorganism, generally a culture is started of blood, urine or tissue samples of the patient in question, it being observed whether metabolic activity of a microorganism occurs therein.

When the culture is “positive”, next the antibiotic susceptibility of the microorganism can be tested, for instance by means of a so-called disk-diffusion test.

To determine the identity of the microorganism, in many cases, isolated colonies need to be obtained; for this purpose, a positive blood culture can be plated on various nutrient media, after which the identity of the microorganism is determined using biochemical tests (see: NCCLS, National Committee for Clinical Laboratory Standards. Approved standard M7-A. Methods for dilution antimicrobial susceptibility tests for bacteria that grow aerobically. National Committee for Clinical Laboratory Standards, Villanova, Pa. 1985).

The methods above take up such an amount of time that most patients, in case of suspected infection, are administered a broad-spectrum antibiotic, that is effective against most known infectious diseases. However, the use of such agents has the disadvantage of inhibiting the growth of substantially all species of microorganisms, including those in the intestinal flora of the patient which are necessary for a good digestion and a good barrier effect against intruding pathogens, with all its additional consequences. Another important disadvantage of a broad-spectrum antibiotic is that the corresponding resistance to the agent develops faster in the population of microorganisms.

For this reason, it is essential that the identity and the antibiotic susceptibility of an infectious microorganism be known as soon as possible to the doctor in attendance, so that a specifically effective antibiotic can be administered.

On the basis of nucleic acid (DNA or RNA) detection techniques, in the past decades, many methods have been developed for the detection and identification of a microorganism, inter alia in a culture of a blood sample. Such methods are aimed at, for instance, detecting a ribosomal RNA gene, or another specific genetic code by which a microorganism can be recognized and identify a microorganism. In this connection, for instance, a nucleic acid amplification reaction, such as a PCR reaction (Mullis, 1987; U.S. Pat. No. 4,683,202) or a NASBA reaction (Compton, J. 1991; WO 91/02818) can be used, often in combination with, for instance, fluorogenic nucleic acid probes to detect the amplified nucleic acid.

In a further improvement of these genetic methods, methods have been developed by which a plurality of specific genes or genetic codes can be determined in one single procedure or reaction, such as, for instance, in a so-called multiplex PCR reaction, making it possible to determine, in addition to the identity, the presence of specific antibiotic resistance genes (see inter alia Maes et al., 2002).

Also, methods are currently known in which oligonucleotide chips or microarrays are used for the simultaneous identification of the microorganism and the detection of antibiotic resistance genes (see inter alia WO 98/20157 and WO 01/92573).

The use of oligonucleotide microarrays makes it possible for large amounts of genetic information to be collected in a very short time. It is inter alia known that DNA microarrays can be used to determine the complete nucleotide sequence of inter alia the rpoB gene, which, if mutated, can cause resistance to rifampicin in mycobacteria.

As discussed above, in the clinic, antibiotic susceptibility and resistance are substantially determined by means of a phenotypic method i.e. by determining a MIC value (minimal inhibiting concentration) using the disk-diffusion method. Antibiotic resistance, on the other hand, can also be tested using a genetic method.

However, to be able to effectively treat infections, for clinical practice, it is inevitable to carry out conventional antibiotic susceptibility determinations based on determination of growth inhibition under the influence of various antibiotics. The fact is, only such tests indicate whether specific treatment methods for reducing the infection will be successful.

Knowledge concerning the presence of one or more resistance genes does not provide the doctor in attendance with information on the agent that is most suitable to suppress and cure an infection in a patient. Also, the genetic method does not provide any quantitative information, such as the degree of insusceptibility.

The DNA techniques currently available do not yet sufficiently meet the need for fast, reproducible identification and the determination of relevant phenotypic characteristics of microorganisms, which is present in laboratory diagnostics and which should be the basis for formulating effective therapy.

The genetic methods currently available for classification of microorganisms practically give a doctor too little information for him to be able to effectively treat a patient.

In particular, there is a lack of insight into relevant clinical data of both microorganism and patient, such as co-medication, age and/or status of the patient or other clinical empirical facts that may be related to the qualitative outcome of the therapy, such as pathology of the strain in question and previous successful and unsuccessful treatment methods.

The present invention provides an apparatus for the classification of microorganism with annotation of clinically relevant information.

An apparatus for classification of a microorganism with annotation of clinically and/or industrially relevant information is capable of meeting the needs referred to above.

An apparatus according to the invention comprises a measuring apparatus with a data file for classifying a microorganism according to distinctive features and a database with clinically and/or industrially relevant information of a plurality of microorganisms, with the measuring apparatus and the database being provided with input and output means and being mutually coupled for the automatic annotation of clinically and/or industrially relevant information to the classified microorganism.

The present invention further provides a method for classifying a microorganism with annotation of clinically and/or industrially relevant information, comprising the use of an apparatus according to the invention.

An apparatus and/or method according to the invention finds highly suitable application in medical practice, such as diagnostic practice aimed at the identification and antibiotic susceptibility determination of clinically relevant microorganisms, prognostic practice aimed at predicting the course of the disease or disorder, and therapeutic practice, aimed at the formulation of an effective treatment plan by a doctor.

Further, an apparatus and/or method according to the invention finds highly suitable application utility in the field of process inspection and process control of industrial microbiological processes, such as food preparation processes and (microbiological) food safety research.

In the present invention, microorganisms are understood to be parasites, fungi, yeasts, bacteria and viruses of various taxonomical levels, such as of different superkingdoms, kingdoms, phyla, classes, orders, families, genera, species or subspecies.

Clinically relevant microorganisms are generally understood to be organisms that can cause an infection in the human body which may or may not be contagious or transmittable. An apparatus and method according to the invention are also suitable for the characterization of viruses in order to be able to prescribe a suitable immunization procedure or anti-viral therapy.

Relevant microorganisms for the food industry are understood to be those microorganisms that originally occur in the required ingredients and/or the food product, or that are capable of surviving the production process and/or the conservation process, and can thus cause a hazard or risk to humans and animals.

An extensive list of clinically relevant microorganisms, which should by no means be construed as limiting, is shown in Table 1, with the organisms being ordered according to taxonomical affiliation.

TABLE 1
Clinically relevant microorganisms

| Viruses | |
|---|---|
| Bacteria Superkingdom | |
| CFB group | |
| Bacteroides (inter alia B. fragilis) | |
| Flavobacterium (inter alia F. meningoseplicum) | |
| Prevotella (inter alia P. intermedia) | |
| Capnocytophaga (inter alia C. canimorsus) | |
| Chlamydiales order | |
| Chlamydia (inter alia C. trachomalis, | |
| C. pneumoniae and C. psittaci) | |
| Fusobacteria | |
| Fusobacterium necrophorum | |
| Streptobacillus moniliformis | |
| Spirochaetales order | |
| Borrelia (inter alia B. burgdorferi and | |
| B. recurrentis) | |
| Treponema (inter alia T. pallidum) | |
| Leptospira (inter alia L. interrogans) | |
| Firmicutes phylum (Gram positive group) | |
| Bifidobacteriales order | |
| Gardnerella (inter alia G. vaginalis) | |
| Lactobacillales order | |
| Streptococcus (inter alia S. pneumoniae, | |
| α-, β- and γ-hemolytic | |
| and viridans and S. pyogenes) | |
| Enterococcus (inter alia E. faecium and | |
| E. faecalis) | |
| Aerococcus (inter alia A. viridans) | |
| Pediococcus (inter alia P. acidilactici) | |
| Leuconostoc (inter alia L. pseudomesenteroides) | |
| Actinomycetales order | |
| Mycobacterium (inter alia M. tuberculosis, | |
| M. lepra, M. africanum, | |
| M. bovis and M. avium) | |
| Nocardia (inter alia N. asteroides) | |
| Corynebacterium (inter alia C. diphtheriae) | |
| Micrococcus (inter alia M. luteus) | |
| Actinomyces (inter alia A. israelii) | |
| Propionibacterium propionicum | |
| Brevibacterium (inter alia B. linens) | |
| Mycoplasmatales order | |
| Mycoplasma (inter alia M. pneumoniae) | |
| Bacillales order | |
| Staphylococcus (inter alia S. aureus | |
| and S. pyogenes) | |
| Alicyclobacillus (inter alia | |
| Alicyclobacillus acidocaldarius) | |
| Listeria (inter alia L. monocytogenes) | |
| Bacillus (inter alia B. anthracis) | |
| Gemella (inter alia G. morbillorum) | |
| Clostridiales order | |
| Clostridium (inter alia C. botulinum, C. diffilie, | |
| C. perfringens and C. tetani) | |
| Peptostreptococcus (inter alia P. prevotii) | |
| Veillonella (inter alia V. parvula) | |
| Proteobacteria phylum | |
| Alpha subdivision Class | |
| Rickettsiales order | |
| Rickettsiaceae family | |
| Rickettsia (inter alia R. prowazekii | |
| and R. typhi) | |
| Ehrlichia (inter alia E canis, E. chaffeensis | |
| and E. phagocytophila) | |
| Cowdria (inter alia C. ruminantium) | |
| Neorickellsia (inter alia N. helminthoeca) | |
| Anaplasma (inter alia A. marginale | |
| and A. ovis) | |
| Wolbachia (inter alia W. pipientis) | |
| Rhizobacteriaceae group | |
| Brucellaceae family | |
| Brucella (inter alia B. melitensis | |
| biovar abortus and B. m. biovar | |
| canis) | |
| Bartonellaceae family | |
| Bartonella (inter alia B. bacilliformis, | |
| B. henselae and B. quintana) | |
| Beta subdidvision Class | |
| Alcaligenaceae family | |
| Alcaligenes (inter alia A. faecalis) | |
| Bordetella (inter alia B. pertussis) | |
| Neisseriaceae family | |
| Neisseria (inter alia N. meningitidis | |
| and N. gonorrhoeae) | |
| Kingella (inter alia K. denitrificans) | |
| Eikenella (inter alia E. corrodens) | |
| Chromobacterium (o., a. C. violaceum) | |
| Burkholderia group | |
| Burkholderia (inter alia B. cepacia) | |
| Gamma subdivision Class | |
| Aeromonadaceae family | |
| Aeromonas | |
| Moraxellaceae family | |
| Acinetobacter (inter alia A. lwoffii and | |
| A. baumannii) | |
| Moraxella (inter alia M. catarrhalis) | |
| Enterobacteriaceae family | |
| Escherichiae (inter alia E. coli) | |
| Klebsiellae (inter alia K. pneumoniae) | |
| Salmonellae (inter alia S. typhimurium | |
| and S. enteritidis) | |
| Shigella (inter alia S. dysenteriac) | |
| Edwardsiella (inter alia E. tarda) | |
| Yersinia (inter alia Y. pestis) | |
| Citrobacter (inter alia C. freundii) | |
| Proteus (inter alia P. mirabilis) | |
| Morganella morganii | |
| Providencia (inter alia P. alcalifaciens) | |
| Serratia (inter alia S. marcescens) | |
| Plesiomonas (inter alia P. shigelloides) | |
| Legionellaceae family/Coxiella group | |
| Legionella (inter alia L. pneumophila | |
| and L. micdadei) | |
| Coxiella (inter alia C. burnetii) | |
| Rickettsiella (inter alia R. popilliae) | |
| Tatlockia (inter alia T. micdadei) | |
| Fluoribacter (inter alia F. dumoffii) | |
| Pasteurellaceae family | |
| Haemophilus (inter alia H. influenzae | |
| and H. ducreyi) | |
| Pasteurella (inter alia P. multocida) | |
| Pseudomonadaceae family | |
| Pseudomonos (inter alia P. aeruginosa | |
| and P. cepacia) | |
| Francisella group | |
| Francisella (inter alia F. tularensis) | |
| Vibrionaceas family | |
| Vibrio (inter alia V. cholerae, V. vulnificus | |
| and V. parahaemolyticus) | |
| Xanthomonas group | |
| Stenotrophomonas (inter alia S. mallophila) | |
| Epsilon subdivision Class | |
| Campylobacter group | |
| Campylobacter (inter alia C. jejuni) | |
| Helicobacter (inter alia H. pylari, H. cinaedi | |
| and H. fennelliae) | |
| Eukaryota Superkingdom | |
| Protista Kingdom | |
| Trichomonadida order | |
| Trichomonas (inter alia T. vaginalis) | |
| Microsporida order | |
| Enterocytozoon bieneusi | |
| Amoebida order | |
| Acanthamoeba (inter alia A. castellani) | |
| Entamoeba (inter alia E. histolytica) | |
| Eucoccidiida order | |
| Cryptosporidium (inter alia C. parvum) | |
| Diplomonadidae order | |
| Giardia lamblia | |
| Eimeriida order | |
| Cryptosporidium (inter alia C. parvum) | |
| Taxoplasma gondii | |
| Neospora caninum | |
| Haemosporida order | |
| Plasmodium (inter alia P. falciparum) | |
| Kinetoplaslida order | |
| Trypanosoma (inter alia T. brucei) | |
| Leishmania donovani | |
| Fungi Kingdom | |
| Acremonium | |
| Aspergillus (inter alia A. fumigatus) | |
| Beauveria | |
| Fusarium | |
| Histoplasma (inter alia H. duboisii) | |
| Paecilomyces | |
| Penicillium | |
| Scopulariopsis | |
| Trichophyton (inter alia T. rubrum and | |
| T. mentagrophytes) | |
| Cryptococcus (inter alia C. neoformans) | |
| Coccidioides (inter alia C. immitis) | |
| Candida (inter alia C. albicans) | |
| Blastomyces | |
| Malassezia | |
| Pneumocystosis (inter alia P. carinii) | |
| Viruses | |
| DNA viruses | |
| Herpesviridae | Herpes simplex virus type 1 |
| | Varicella zoster virus |
| | Epstein Barr virus |
| | Human cytomegalovirus |
| | Human herpesvirus 6 |
| Adenoviridae | Human adenoviruses |
| Papovaviridae | Human papillamaviruses |
| Hepadnaviridae | Hepatitis B virus |
| Poxviridae | Vaccinia virus |
| Parvoviridae | B19 parvovirus |
| RNA viruses | |
| Picornaviridae | Polioviruses |
| | Echoviruses |
| | Coxsackieviruses |
| | Hepatitis A virus |
| | Human rhinoviruses |
| Caliciviridae | Norwalk virus |
| Paramyxoviridae | Parainfluenza viruses |
| | Measles virus |
| | Respiratory syncytial virus |
| Orthomyxoviridae | Influenxa virus |
| Rhabdoviridae | Rabies virus |
| Filoviridae | Ebola and Marburg viruses |
| Retroviridae | Human immunodeficiency |
| | virus type-1 and -2 |
| Togaviridae | Rubella virus |
| Flaviviridae | Yellow fever virus |
| | Dengue virus |
| Reoviridae | Human rotaviruses |
| Bunyaviridae | Pulmonary Syndrome |
| | Hantavirus |
| | Hantaan virus |
| Arenaviridae | Lassa virus |
| Coronaviridae | Human coronaviruses |
| Astroviridae | Human astroviruses |

Karolinska Instituted Library Bacterial Infections and Mycoses (www.mic.ki.se); Atlas of Medical Parasitology, Carlo Denegri Foundation (www.cdfound.to.it); NCBI taxonomy database (www.ncbi.nlm.nih.gov); University of Rochester Medical Center Dept. of Microbiology and Immunology (www.urmc.rochester.edu)

A measuring apparatus for classifying a microorganism according to distinctive features according to the invention can comprise a single or multiple measuring apparatus.

For classifying an organism, preferably a measuring apparatus is used in the invention which can be used to measure morphologically, physiologically, serologically, pathologically, taxonomically and/or genetically distinctive features between microorganisms.

In the context of the present invention, a morphological feature of a microorganism is understood to refer to an externally observable feature such as the form of the organism; the possession of a specific biochemical substance, for instance a membrane peptide, a pigment, a (glyco)protein, a lipid or a cell wall component such as mycolic acid; the possession or absence of a specific receptor; the production of spores or cysts; the possession of flagella; growing in chains or in filaments, or another external feature such as a cell or colony morphology; or a coloring characteristic.

In the context of the present invention, a physiological feature of a microorganism is meant to refer to a specific catabolic characteristic such as proteolysis or a capability to grow on specific substrates such as polysaccharides, proteins, fats or nucleic acids; specific nutrient requirements; the possession of specific metabolic routes; a sensitivity to oxygen or a susceptibility to an antibiotic; a temperature or acidity dependence; the production of a specific metabolic final product; the secretion of a bacteriocin or antibiotic; the production of a gas; the manner of energy supply of the organism; the size, composition or another feature of the collection of proteins in the cell (the proteome); or a feature of the collection of low-molecular organic substances in the cell (the metabolome).

In the context of the present invention, a serological feature is meant to refer to the capability to react with a specific antibody or monoclonal; the possession or absence of specific surface antigens or epitopes such as glycolipids or glycoproteins.

In the context of the present invention, a pathological feature of a microorganism is meant to refer to a capability to infect cells; toxin secretion; a manner in which an infection progresses; a hemolytic characteristic or other pathological feature, such as the natural habitat or the tissue or cell type that is affected by the organism.

In the context of the present invention, a taxonomical feature is defined as a phenotypic feature, such as a morphological feature, a physiological feature, a serological or pathological feature as described above, on the basis of which a microorganism is usually taxonomically identified, but can also comprise a genetic feature on the basis of which the phylogenetic lineage can be determined, and on the basis of which a microorganism can also be taxonomically identified.

In the context of the present invention, a genetic feature is meant to refer to a specific chromosomal or extra-chromosomal distinctive nucleotide sequence of a nucleic acid such as a DNA and/or an RNA; a specific genetic code or a gene; a linear or circular chromosome; the size or another feature of the genome; the G+C content; the presence of plasmids; the possession of specific transposons, integrons or insertion sequences; the composition or size of the expression profile (transcriptome).

A measuring apparatus according to the invention for classifying a microorganism according to distinctive features can be arranged such that it can measure different or the same types of distinctive features between microorganisms, such as morphological, physiological, serological, pathological, taxonomical and/or genetic features.

If different types of distinctive features between microorganisms can be measured using an apparatus according to the invention, these features can be measured sequentially and/or simultaneously.

A measuring apparatus according to the invention can inter alia be used to determine a genetically distinctive feature of a microorganism. Such a genetically distinctive feature can comprise, for instance, a resistance gene to which a specific antibiotic-resistant phenotype is related. This can be understood to refer to inter alia the mecA gene encoding the penicillin-binding protein 2a in Staphylococcus, making this bacterium insusceptible to substantially all β-lactam antibiotics, including meticillin.

Other resistance genes that can be measured using a measuring apparatus according to the invention that can measure a genetically distinctive feature of a microorganism, are inter alia the aac(6′) gene in Serratia marcescens or Klebsiella pneumoniae, which causes resistance to aminoglycosides, such as netilmicin and gentamicin, or resistance genes such as nptII (kanamycin resistance), vanA, B and C (vancomaycin resistance), ermA, B and C msrA (macrolide resistance), gyrA, grlA (quinolone resistance), bla (β-lactam resistance), vat, vga (streptogramin resistance), or sul and int (sulfonamide resistance).

In addition to measuring specific resistance genes, a measuring apparatus according to the invention which can measure a genetically distinctive feature of a microorganism can also be used to measure mutations in specific genes. Mutations in the PfCRT transmembrane protein of the digestive vacuole or in the PfMRD1 gene for the P-glycoprotein homolog 1 (Pgh1) of Plasmodium falciparum cause, for instance, insusceptibility of this parasite to agents such as chloroquine and can be measured using a measuring apparatus according to the invention.

In an alternative embodiment, mutations in, for instance, a ribosomal RNA gene can be detected, on the basis of which taxonomical or phylogenetic information and/or features can be determined.

Also, other genes or genetic codes, the function of which is yet is unknown, can provide relevant distinctive features between microorganisms which can be measured in an embodiment according to the present invention, as long as they represent a distinctive feature for a classification of a microorganism according to the invention.

For measuring distinctive features in a microorganism using a measuring apparatus according to the invention, preferably markers are used in an apparatus according to the invention.

In the present invention, a marker is defined as a characteristic distinctive feature of a microorganism that can be measured, preferably by using molecular biological methods, for instance by determining or measuring the distinctive feature by using complementary binding partners, such as complementary nucleic acids or complementary oligonucleotide probes in the case of a genetic feature, or an antibody or monoclonal or another binding partner in the case of a physiological feature such as a protein. Suitable markers or binding partners are assumed to be known to a skilled person. The detection of binding between the complementary binding partner and the marker can be facilitated by using labels.

When in the present invention nucleic acid markers or genetic markers are referred to, this is understood to mean complementary binding partners as well.

Markers can comprise genetic or phenotypic markers. Preferably, in a measuring apparatus according to the present invention, genetic and/or physiological markers, such as protein markers, are used. With great preference, genetic markers are used.

In addition to the fact that genetic markers can be used in a measuring apparatus according to the present invention for determining the presence of (known) genetic distinctive features in a microorganism, they can also be used highly suitably to determine phenotypic features, such as for instance antibiotic susceptibility and/or antibiotic resistance.

To detect relevant genetic and/or phenotypic features, genetic markers can be used highly suitably in an apparatus according to the present invention. Many genetic markers that are suitable for use in an apparatus according to the invention are known to a skilled person. A skilled person can also identify and produce suitable genetic markers himself in a simple manner.

Methods for identifying genetic markers related to specific phenotypic characteristics of microorganisms are known to a skilled person (see inter alia WO 01/83813). For instance, so-called polymorphisms can be detected using methods described in U.S. Pat. No. 6,300,063. For this purpose, known genetic fingerprint methods can also be used (see Mueller and Wolfenbarger for an overview), such as AFLP (Vos et al., 1995), RAPD, or RFLP (Botstein et al., 1980) or techniques derived therefrom such as Ribotyping.

In addition to the fact that such fingerprint methods can be used to identify genetic markers, they can also result in a fingerprint which can be used to distinguish microorganisms. As such, these fingerprint methods and the instruments and equipment available for these find suitable application utility in an apparatus and method according to the invention for classifying a microorganism according to distinctive features.

In the present invention, genetic markers such as RFLP markers (see, for instance, U.S. Pat. No. 5,324,631), RAPDs (see, for instance, Aufauvre-Brown et al., 1992), AFLP markers (see, for instance, EP 0,534,858) SSR markers (see, for instance, U.S. Pat. No. 5,075,217) and SNP markers McEwen et al., 2000) can be used.

A method comprising the use of an apparatus according to the invention in an embodiment in which genetic markers are used, preferably comprises a step for determining the presence or absence of a specific distinctive feature or a marker in a nucleic acid, such as a DNA or RNA, of a microorganism, for instance by means of fingerprinting or using a microarray, such as a DNA array, an oligonucleotide array or, in general terms, a nucleic acid array.

In a preferred embodiment, the invention relates to an apparatus in which a nucleic acid array is used, which comprises oligonucleotides or nucleic acid sequences of genetic markers or complements thereof immobilized on a carrier surface.

Genetic markers can also be used for the amplification of nucleic acid sequences which are associated to a specific distinctive feature, in the present case as, for instance, primers. Methods to characterize such amplified products, such as electrophoresis, chromatography, sequencing or mass spectrometry, are known to a skilled person.

To improve hybridization characteristics of oligonucleotides or nucleic acid sequences, specific nucleic acid analogs can be used which can enter into a sequence-specific interaction equal to that of the natural phosphodiester nucleic acid, such as phosphorothioate or methylphosphonate oligonucleotides or peptide nucleic acid (PNA) oligonucleotides.

In a method according to the present invention, use of a DNA array is preferred. Such arrays of oligonucleotides then comprise the complementary binding partners of genetic markers, and are also part of the present invention.

The production of an oligonucleotide array according to the invention can be carried out using methods known to a skilled person. The production and the use of solid-carrier nucleic acid arrays for the detection of specific nucleic acid sequences has frequently been described (U.S. Pat. No. 5,571,639; Sapolsky et al., 1999, Genet. Anal.-Biomolecular Eng. 14, 187-192; Shena et al., 1995, Science 270, 467-470; Sheldon et al., 1993, Clinical Chem. 39, 718-719; Fodor et al., 1991, Science 251, 767-773).

A skilled person will be capable of obtaining arrays by his own design and the corresponding array reading equipment from specialized suppliers (for instance Affymetrix Corp., Santa Clara, Calif., USA for DNA arrays and Ciphergen Biosystems, Fremont, Calif., USA for protein arrays).

A DNA array according to the present invention can comprise, for instance, between 10 and 200,000 oligonucleotides which are specific for specific sequences in the form of genetic markers. Also, an array can comprise oligonucleotides which comprise genetic markers such as SNPs and microsatellite markers. Methods for designing sets of oligonucleotide probes for simultaneous analysis of nucleic acids, such as expression products of genes, are described in inter alia EP 0,799,897.

It is advantageous when the melting point of the oligonucleotides is substantially in the same range to enable hybridization in uniform conditions.

In particular, synthesis of the oligonucleotides can be carried out directly on the solid carrier surface of the array, such as for example using a photochemical synthesis technique described in U.S. Pat. No. 5,424,186 or using an ink-jet technique. In an alternative embodiment, the oligonucleotides can be synthesized ex situ and bound to the solid carrier surface. In this case, it is advantageous if the carrier surface has been chemically modified prior to the application of the oligonucleotides to enable binding between the oligonucleotides and the carrier surface, optionally using a hydrogel matrix or additional organic or inorganic linkers between the oligonucleotide and the carrier surface of the array. Addressing the various oligonucleotides on the surface can be done electronically, mechanically or using an ink-jet.

The hybridization conditions will depend on the nucleic acid used as sample material, but can be optimized in a simple manner using methods known to a skilled person. For this purpose, inter alia the salt content, the pH and the temperature of the hybridization can be adjusted. Optionally, methods can be used to electronically control the stringency of the hybridization, as known from U.S. Pat. No. 6,017,696.

The detection of the hybridization spots can be cared out using labels such as radioisotope labels or fluorescent labels, using field effect measurements, using optoelectrochemical methods, piezo-electric methods, or ellipsometry, measurement using optical fibers or mass spectrometry. Also, telemetry can be used to investigate the presence of markers in the starting nucleic acid.

Prior to hybridization, the nucleic acid fragments can highly suitably be labeled, for instance using a fluorescent label or a radioisotope or another label, to facilitate the detection of these fragments hybridized to the oligonucleotides on the array. Depending on the selected method of detection, a skilled person will be able to use a suitable label.

A method comprising the use of an apparatus according to the invention preferably comprises a step for determining the presence or absence of a specific distinctive feature in a microorganism, in which a sample containing material of the microorganism is brought into contact with the measuring apparatus. Such a contact location can very suitably serve as input means for the measuring apparatus.

The apparatus according to the invention comprises a measuring apparatus with a data file for classifying a microorganism according to distinctive features. Such a data file preferably comprises data concerning markers and distinctive features of microorganisms as described above. The measuring apparatus will further comprise a calculation unit to process the measuring results obtained by using the measuring apparatus.

In an embodiment of the method according to the invention in which a nucleic acid array is used as contact location of a measuring apparatus, nucleic acid of a microorganism or fragments thereof are brought into contact with the array of nucleic acid markers.

Methods for obtaining genetic information using nucleic acid arrays are known in the literature (see inter alia Chee et al., 1996).

A measuring apparatus for measuring distinctive features in a microorganism according to the invention can, in an alternative embodiment, be formed by, for instance, an apparatus for “Matrix Assisted Laser Desorption/Ionization-Time of Flight Mass Spectrometry” (MALDI-TOF-MS), “Surface-Enhanced Laser Desorption/Ionization Time-of-Flight Mass Spectrometry” (SELDI-TOF-MS), “High Performance Liquid Chromatography tandem Mass Spectrometry” (HPLC-MS/MS). “surface plasmon resonance” (SRP), optionally using markers.

A measuring apparatus according to the present invention can be arranged for single or plural measurements, but is preferably arranged for plural (simultaneous) measurements. A measuring apparatus according to the invention is further preferably arranged for receiving, handling, processing, classifying, categorizing, filing, outputting and/or storing results of measurements of distinctive features for classifying a microorganism according to the invention and is, for this purpose, preferably provided with means such as a calculation unit and a data file, which data file comprises distinctive features of microorganisms for the classification of a microorganism.

A measuring apparatus with a data file of distinctive features is preferably also arranged for filing measuring results obtained by using a measuring apparatus according to the invention.

A calculation unit can be used as part of the measuring apparatus, but can also be used externally or separate from the measuring apparatus. A calculation unit is arranged for performing calculations, in which results obtained using a measuring apparatus according to the invention can be compared to distinctive features in a data file. For this purpose, a calculation unit preferably comprises input and output means that can be connected to corresponding output means of a measuring apparatus and corresponding input and output means of a data file with distinctive features.

Further, a calculation unit according to the invention is preferably arranged for categorizing and classifying distinctive features of microorganisms obtained using a measuring apparatus according to the invention according to taxonomical position of a microorganism. For this purpose, preferably, the results are sent as data from a measuring apparatus according to the invention to an input of a calculation unit according to the invention, to be subsequently categorized or classified by a calculation unit according to the invention.

A calculation unit according to the invention is preferably a mathematical calculation unit for solving algorithmic comparisons in which categorized and classified distinctive features of microorganisms are inter alia statistically compared to relevant information from a database and in which a calculation result is outputted, preferably to a display. Preferably, a calculation unit according to the invention comprises a microprocessor unit.

With great preference, for comparison analyses by a calculation unit, algorithmic computer analysis methods are used such as self-organizing maps, hierarchic clustering, multidimensional scaling, principal component analysis, supervised learning, k-nearest neighbors, support vector machines, discriminant analysis or partial least square methods. Such methods are known to a skilled person.

A calculation unit according to the invention is arranged for classifying a microorganism according to distinctive features. Preferably, this classification comprises a taxonomical classification. With great preference, the classification results in a detailed taxonomical positioning or identification of a microorganism.

An apparatus for classifying a microorganism with annotation of clinically and/or industrially relevant information according to the invention further comprises a database containing clinically and/or industrially relevant information of a plurality of microorganisms and preferably for a plurality of clinical and/or industrial situations.

A database containing clinically and/or industrially relevant information according to the invention comprises at least one memory location for storage of all possible types of information which are in any way related to microorganisms that play a role in a clinical and/or industrial environment, preferably digitized. A database according to the invention can comprise a combination of clinically relevant information and industrially relevant information. Preferably, a database according to the invention comprises relevant information for one of the two environments, with this information being limited to a specific use of the apparatus according to the invention.

Information that is in any way related to microorganisms that play a role in a clinical environment is preferably categorized according to different taxonomical groups and based on different taxonomical levels of microorganisms. Furthermore, information may be categorized according to different natural or artificial, known or uncharacterized mixed populations of microorganisms.

Such information can, for instance, comprise information concerning the microorganisms themselves and the nature of the infection they cause, such as geographical origin of the microorganism, the incubation time in which disease symptoms become manifest after exposure, the antibiotics of which an effect on the microorganism is known, from information concerning patients who suffer or have suffered from the infection, such as average age, the co-medication, the health status, the ethnic origin, the epidemiological origin, the family relationship, etc., and/or from information concerning treatment methods which have already been used for specific infections, such as medication, diet, relation food/environment.

Information that is in any way related to microorganisms that play a role in an industrial environment can, for instance, comprise information concerning the microorganisms and the nature of the process they carry out, such as information concerning physical and biological process parameters, such as pH, Aw (water activity) and the temperature sensitivity and/or information concerning process treatments which have already been used for specific industrial processes, such as cooling, freezing, pasteurizing, sterilizing, but also alternative techniques such as the use of high pressure, light, electric or magnetic fields and radiation. Also, this may involve effects resulting from the effects of cleaning and the use of disinfectants.

A database according to the invention is provided with a means intended for data input and is preferably connected to a data presentation unit by means of an output.

It is possible to add new information to the database or to remove information therefrom, which yields a database with a dynamic character. By adding new relevant information, the size and the detail of the database will increase, thus providing an increasingly better basis for results.

A database containing clinically or industrially relevant information according to the invention and a data file containing distinctive features as part of a measuring apparatus for classifying a microorganism according to the invention can be combined in an alternative embodiment.

Annotation of clinically or industrially relevant information which is present in a database according to the invention is done by combining the information of the classification of the microorganism or of the microorganisms obtained using the measuring apparatus according to the invention with the relevant information from the database for the microorganism in question or a higher taxonomical level thereof.

The output of an apparatus according to the invention can, for instance, take place in the form of a chance that a specific proposed therapy or process treatment method will be successful, or can, for instance, take place in the form of a proposal for a highly suitable therapy or process treatment method.

References

Aufauvre-Brown A., Cohen J., Holden D W. 1992. Use of randomly amplified polymorphic DNA markers to distinguish isolates of Aspergillus fumigatus. J Clin Microbiol. 30(11):2991-3.
Botstein D, White R L, Skolnick M, Davis R W. 1980. Construction of a genetic linkage map in man using restriction fragment length polymorphisms. Am J Hum Genet. 32(3):314-31.
Chee M, Yang R, Hubbell E, Berno A, Huang X C, Stern D, Winkler J, Lockhart D J, Morris M S, Fodor S P. 1996. Accessing genetic information with high-density DNA arrays. Science 274(5287):610-4.
Compton, J. (1991). Nucleic acid sequence-based amplification. Nature 350 (6313), 91-2.
Davies, J. (1997). Origins, acquisition and dissemination of antibiotic resistance determinants. In: Antibiotic Resistance: Origins, Evolution, Selection and Spread. Ciba Foundation Symposium 207, S. 15-35, Wiley, Chichester.
Fodor, S. P. A. Read, J. L. Pirrung, M. C. Stryer, L. Lu A. T. and Solas D. 1991. Light-Directed, Spatially Addressable Parallel Chemical Synthesis. Science 251:767-773.
Maes N., Magdalena J., Rottiers S., De Gheldre Y. and Struelens M. J. (2002). Evaluation of a Triplex PCR Assay To Discriminate Staphylococcus aureus from Coagulase-Negative Staphylococci and Determine Methicillin Resistance from Blood Cultures. J Clin Microbiol. 40 (4):1514-1517.
McEwen J G, Taylor J W, Carter D, Xu J, Felipe M S, Vilgalys R, Mitchell T G, Kasuga T, White T, Bui T, Soares C M. Molecular typing of pathogenic fungi. Med Mycol. 2000;38 Suppl 1: 189-197.
Mullis, K. B., and Faloona, F. A. (1987). Specific synthesis of DNA in vitro via a polymerase-catalyzed chain reaction. Methods Enzymol., 155, 335-50.
Vos, P., Hogers, M., Bleeker, M., Reijans, M., van de Lee, T., Hornes, M., Frijters, A., Pot, J., Peleman, J., Kuiper, M. and Zabeau, M. (1995). AFLP: a new technique for DNA fingerprinting. Nucleic Acids Res., 23, 4407-4414.
Mueller, U. G. and Wolfenbarger, L. L. (1999). AFLP genotyping and fingerprinting—a review. Trends in Ecol. & Evol., 14, 389-394.

1. An apparatus for classification of a microorganism with annotation of clinically and/or industrially relevant information, comprising a measuring apparatus containing a calculation unit and a data file for classifying a microorganism according to distinctive features and a database containing clinically and/or industrially relevant information of a plurality of microorganisms, wherein said measuring apparatus and said database are provided with input and output means and are mutually coupled for the automatic annotation of said clinically and/or industrially relevant information to the classified microorganism.

2. An apparatus according to claim 1, wherein said microorganisms comprise clinically relevant microorganisms.

3. An apparatus according to claim 1, wherein said measuring apparatus comprises an array of physiological and/or genetic markers.

4. An apparatus according to claim 3, wherein said genetic markers comprise oligonucleotides.

5. An apparatus according to claim 1, wherein the data file of said measuring apparatus comprises morphological, physiological, serological, pathological, taxonomical or genetic features.

6. An apparatus according to claim 1, wherein said database comprises clinically relevant information and/or industrially relevant information.

7. An apparatus according to claim 6, wherein said clinically relevant information comprises information concerning microorganisms, concerning the nature of an infection, concerning patients and/or concerning treatment methods.

8. An apparatus according to claim 6, wherein said industrially relevant information comprises information concerning microorganisms, concerning the nature of a process, concerning process parameters and/or concerning process treatments.

9. An apparatus according to claim 1, wherein said calculation unit is arranged for performing algorithmic computer analyses selected from the group consisting of self-organizing maps, hierarchic clustering, multidimensional scaling, principal component analysis, supervised learning, k-nearest neighbors, support vector machines, discriminant analysis and partial least square.

10. An apparatus according to any claim 1, wherein said calculation unit is arranged for categorizing and classifying distinctive features of microorganisms obtained by using a measuring apparatus according to the invention.

11. A method for classifying a microorganism with annotation of clinically and/or industrially relevant information, comprising the use of an apparatus according to the invention.

12. A method according to claim 11, wherein a sample containing material of a microorganism is brought into contact with a measuring apparatus according to the invention at a contact location intended for the purpose to measure the presence or absence of a particular distinctive feature in a microorganism.
